(19) United States
(12) Patent Application Publication
(10) Pub. No.: US 2026/0260722 A1
(43) Pub. Date: Sep. 3, 2026

(54) SYSTEM AND METHOD FOR REESTABLISHING COMMUNICATION IN WEARABLE MEDICAL SYSTEMS

(71) Applicant: West Affum Holdings DAC, Dublin (IE)

(72) Inventors: Brian D. Webster, Mercer Island, WA (US); Jeffrey M. Boschee, Woodinville, WA (US); Gordon P. Teddy, Maple Valley, WA (US)

(73) Assignee: West Affum Holdings DAC, Dublin (IE)

(21) Appl. No.: 19/542,293

(22) Filed: Feb. 17, 2026

Related U.S. Application Data
(60) Provisional application No. 63/761,107, filed on Feb. 20, 2025.

(51) Int. Cl. G16H 10/60; G16H 40/67

(52) U.S. Cl. CPC G16H 10/60; G16H 40/67

(57) ABSTRACT

A medical system for reestablishing communication in wearable medical systems is disclosed. The medical system includes a wearable medical device, one or more remote servers, and a mobile device comprising at least one processor and a user interface, the mobile device configured to be in communication with the wearable medical device and the one or more remote servers. The at least one processor establishes a wireless connection between a patient application of the mobile device and the wearable medical device. Further, the at least one processor transmits the patient data to the one or more remote servers when the wireless connection between the mobile device and the wearable medical device is established. Furthermore, the at least one processor is configured to receive a communication from the one or more remote servers when the patient data is not transmitted to the one or more remote servers during inactivity.

### CROSS-REFERENCE TO RELATED APPLICATIONS

This application claims priority to and the benefit of the provisional patent application No. 63/761,107 titled "WCD SYSTEM WITH ASSISTANT DEVICE RECEIVING TEXT NOTIFICATION TO RELAUNCH DOWNLOADED APP WHEN APP IS SUSPENDED," filed in the United States Patent and Trademark Office on February 20, 2025. The specification of the above referenced patent application is incorporated herein by reference in its entirety.

### TECHNICAL FIELD

The present technology relates to the field of wearable medical systems, and more particularly, but not by way of limitation, relates to a system and method for reestablishing communication between a wearable medical device and a remote monitoring system, and for prompting a patient to restore or maintain such communication.

### BACKGROUND

In wearable medical systems, patients often use devices such as wearable cardiac monitors, fitness trackers, or other health monitoring instruments that continuously collect physiological or biometric data. These wearable medical devices transmit collected data to a remote monitoring system or cloud-based server, typically through a smartphone or other intermediary device. Continuous and reliable communication between the wearable medical device and the monitoring system ensures accurate health tracking, timely alerts, regulatory compliance, and adherence to prescribed treatment or usage protocols. Interruptions in this communication can compromise patient monitoring, reduce data completeness and reliability, and impede healthcare providers' ability to make informed diagnostic or therapeutic decisions.

However, existing wearable medical systems face limitations in addressing unexpected interruptions caused by smartphone operating system restrictions, application suspension, user settings, or other environmental factors. In many cases, the patient may not be actively alerted to restart or maintain the application or communication link, resulting in extended periods of lost data and delayed interventions. Current systems may also lack intelligent or adaptive scheduling of notifications based on patient behavior, device usage patterns, or activity trends, potentially reducing the effectiveness of alerts. Accordingly, there remains a need for improved systems and methods that can detect communication interruptions, assess their likely causes, and actively prompt the patient to restore or maintain connectivity in a timely and context- aware manner.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

According to an embodiment of the present disclosure, a medical system is disclosed. The medical system includes a wearable medical device configured for at least one of collecting patient data related to detection of a cardiac condition or providing therapy to a patient upon detection of the cardiac condition, one or more remote servers, and a mobile device including at least one processor and a user interface. The mobile device is configured to be in communication with the wearable medical device and the one or more remote servers. The at least one processor is configured to establish a wireless connection between the mobile device and the wearable medical device. In addition, the at least one processor is configured to transmit, via the mobile device, the patient data to the one or more remote servers when the wireless connection between the mobile device and the wearable medical device is established. Further, the at least one processor is configured to receive a communication from the one or more remote servers when the patient data has not been transmitted to the one or more remote servers for a period of inactivity. The communication is configured to provide one or more instructions to the patient to perform one or more actions to reestablish the wireless connection between the mobile device and the wearable medical device.

A further embodiment of the present disclosure includes a method for reestablishing a wireless connection between a mobile device and a wearable medical device, the method including above-mentioned operations.

A further embodiment of the present disclosure includes non-transitory computer readable medium, encoded with instructions stored thereon for reestablishing a wireless connection between a mobile device and a wearable medical device, that when executed by at least one computing device, cause the at least one computing device to perform the above-mentioned operations for reestablishing the wireless connection.

### BRIEF DESCRIPTION OF THE DRAWINGS

The above-mentioned implementations are further described herein with reference to the accompanying figures. It should be noted that the description and figures relate to exemplary implementations and should not be construed as a limitation to the present disclosure. It is also to be understood that various arrangements may be devised that, although not explicitly described or shown herein, embody the principles of the present disclosure. Moreover, all statements herein reciting principles, aspects, and embodiments of the present disclosure, as well as specific examples, are intended to encompass equivalents thereof.

Example embodiments will be described and explained with additional specificity and detail through the use of the accompanying drawings in which:

FIG. 1 illustrates a medical system for reestablishing communication between a wearable medical device and a mobile device, in accordance with an embodiment of the present disclosure;

FIG. 2 illustrates an exemplary environment for implementing time-adaptive communication control in a medical system for reestablishing communication between a wearable medical device and a mobile device, in accordance with an embodiment of the present disclosure;

FIG. 3 illustrates an exemplary environment to detect notification settings and transmit communication prompts to a patient application of a mobile device, in accordance with an embodiment of the present disclosure;

FIG. 4 is a flowchart illustrating an exemplary operational sequence for reestablishing communication between a wearable medical device and a mobile device, in accordance with an embodiment of the present disclosure;

FIG. 5 is a block diagram illustrating components of a wearable medical device, in accordance with an embodiment of the present disclosure; and

FIG. 6 illustrates a method for reestablishing communication in a wearable medical system, in accordance with an embodiment of the present disclosure.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, and the like. In other instances, well-known structures or methods, associated with an authorization system for securing protected health information, have not been shown or described in detail to avoid unnecessarily obscuring descriptions of the embodiments.

Unless the context indicates otherwise, throughout the disclosure and claims which follow, the word "comprise" and variations thereof, such as, "comprises" and "comprising" are to be construed in an open, inclusive sense that is as "including, but not limited to." Further, the terms "first," "second," and similar indicators of the sequence are to be construed as interchangeable unless the context clearly dictates otherwise.

Reference throughout this disclosure to "one aspect" or "an aspect" means that a particular feature, structure, or characteristic described in connection with an embodiment is included in at least one aspect. Thus, the appearances of the phrases "in one aspect" or "in an aspect" in various places throughout this disclosure are not necessarily all referring to the same embodiment. Furthermore, the particular features, structures, or characteristics may be combined in any suitable manner in one or more aspects.

As used in this disclosure and the appended claims, the singular forms "a," "an," and "the" include plural referents unless the content clearly dictates otherwise. It should also be noted that the term "or" is generally employed in its broadest sense, that is, as meaning "and/or" unless the content clearly dictates otherwise.

In the interest of not obscuring the presentation of embodiments of the present disclosure, in the following detailed description, some processing steps or operations that are known in the art may have been combined together for presentation and for illustration purposes and in some instances may have not been described in detail. In other instances, some processing steps or operations that are known in the art may not be described at all. It should be understood that the following disclosure is rather focused on the distinctive features or elements of various embodiments of the present disclosure.

Throughout the specification, the term "wearable medical device" may refer to any medical apparatus configured to be worn by a patient and capable of monitoring one or more physiological parameters, delivering therapy, or both. The wearable device can be configured to only monitor one or more physiological parameters (e.g., collect patient data related to a cardiac condition), only deliver therapy upon detection of a cardiac condition, or both. In certain embodiments, the wearable medical device may include sensors, electrodes, or detectors configured to collect physiological data such as electrocardiographic signals, heart rate, respiratory activity, or motion data. The wearable medical device may also include one or more therapy electrodes in communication with an energy source, configured to deliver therapeutic energy such as a defibrillation shock or electrical stimulation in response to detection of a cardiac condition or other health event based on the patient data. The wearable medical device may further include a communication module, such as a Bluetooth or Wi-Fi transceiver, enabling wireless data transmission to a paired mobile device.

Throughout the specification, the term "mobile device" may refer to any portable electronic device operated by or provided to a patient, configured to communicate wirelessly with the wearable medical device and one or more remote servers. Examples of mobile devices may include smartphones, tablets, or other computing devices including at least one processor, memory, and a user interface.

Throughout the specification, the term "patient application" may refer to a patient- facing software application or a software module or an executable application installed or accessible on the mobile device and configured to manage communication between the mobile device and the wearable medical device. The patient application may include routines for establishing or maintaining a wireless connection, transmitting patient data to one or more remote servers, and receiving communications or notifications. The patient application executing on the mobile device may provide a user interface configured to present messages, alerts, and instructions that guide the patient to perform actions such as reestablishing communication connectivity, restarting the application, enabling notification permissions, or adjusting device or application settings.

Throughout the specification, the term "remote server" may refer to one or more computing systems located remotely from the patient and configured to receive, process, and store patient data transmitted from the mobile device. The remote server(s) may be implemented as cloud-based or distributed computing platforms, each including a communication interface, data processing modules, and databases for storing patient-specific data, system activity logs, and operational metrics. The remote server(s) may also be configured to analyze transmission patterns, detect periods of inactivity, and generate communications or instructions to be sent to the mobile device.

Throughout the specification, the term "patient data" may refer to any physiological, behavioral, or operational data generated, collected, or transmitted by the wearable medical device or the mobile device. Patient data may include, without limitation, biometric measurements, cardiac signals, accelerometer data, device usage logs, communication status, notification settings, and sleep or activity indicators. The patient data may be stored temporarily on the mobile device and periodically transmitted to one or more remote servers via secure communication channels.

Throughout the specification, the term "period of inactivity" may refer to a duration of time during which patient data is not transmitted from the mobile device to the one or more remote servers. The period of inactivity may be determined relative to expected transmission intervals defined by the patient application or system configuration. In some embodiments, the remote server may detect a period of inactivity that exceeds a threshold interval and initiate the transmission of communication from the remote server to the mobile device.

Throughout the specification, the term "notification setting" may refer to one or more configuration parameters within the patient application that governs whether alerts, messages, or other notifications are permitted to be presented, displayed, or sounded on the mobile device. The notification setting may be represented as a binary on/off state or may include more granular preferences, such as vibration-only alerts, audible alerts, visual alerts, priority-based notifications, or scheduled quiet hours. In certain embodiments, a remote server may detect that the notification setting is disabled or otherwise restricts notification delivery and may transmit a secondary communication instructing or prompting the patient to modify the notification setting.

Throughout the specification, the term "patient data module" may refer to a logical or software component of the remote server configured to analyze patient data, determine patterns of activity or inactivity, and generate operational instructions based on such determinations. The patient data module may determine a patient's sleep schedule or other behavioral patterns and may suppress, prevent, or delay transmission of communications during periods in which the patient is likely asleep or inactive.

Throughout the specification, the term "user interface" may refer to any visual, auditory, or tactile interface element of the mobile device or wearable medical device through which a patient interacts. The user interface may include graphical screens, touchscreen elements, buttons, display indicators, or audio prompts configured to convey information or instructions to the patient. In certain embodiments, the user interface of the mobile device may display communications or alerts received from the remote server and guide the patient through the reconnection process.

Throughout the specification, the term "therapy" may refer to any treatment or intervention delivered by the wearable medical device to the patient upon detection of a medical condition. Examples of therapy may include delivery of electrical energy for cardiac defibrillation, pacing, or other therapeutic stimulation intended to treat or prevent cardiac events. The delivery of therapy may be triggered automatically by detection algorithms or manually under specific conditions.

Throughout the specification, the term "computing device" may refer to any hardware or software system comprising one or more processors, memory, and communication interfaces configured to execute instructions, process data, and control operations associated with the wearable medical system. Examples of computing devices include mobile devices, remote servers, base stations, or cloud-based computing resources.

Various wearable medical systems currently implement mechanisms to maintain connectivity with the mobile devices and/or remote monitoring systems (e.g., one or more remote servers). Such mechanisms include, but may not be limited to, automated reconnection protocols, notifications from smartphone applications, and routine checks to detect lost or disrupted communication links. Some systems employ periodic data polling, Bluetooth reconnection attempts, or prompts from an associated mobile application to reestablish data transmission. These measures aim to mitigate communication gaps and maintain continuous monitoring of patient health parameters.

In certain wearable medical systems, a wearable medical device communicates with a remote monitoring platform, such as a server or cloud-based system, through an application (or patient application) executing on a mobile device. The communication link between the wearable medical device and the mobile device may be established using one or more wireless communication protocols and may support the transmission of physiological data, device status information, or other operational data.

Under certain conditions, the communication link between the wearable medical device and the mobile device may become inactive, suspended, or otherwise interrupted. Such interruptions may occur, for example, due to a lack of data exchange over the communication link, operating system power-management policies, application lifecycle constraints, or other device- related or environment-related factors. In some instances, an application executing on the mobile device may enter a background, suspended, or inactive state, which may prevent wireless communication events from being delivered to the application, limit the application's ability to respond to such events, or otherwise cause the communication link to be disrupted.

When the application transitions from an inactive state to an active state, the application may be required to reestablish the wireless communication link in order to restore communication between the wearable medical device and the mobile device. However, the operating system of the mobile device may impose time constraints or other limitations on reestablishing such wireless connections during certain application state transitions. In some cases, these constraints may hinder successful reconnection. In certain situations, reestablishing the wireless communication link may require restarting or relaunching the application on the mobile device. However, the patient or user may not be aware that the communication link has been interrupted and, as a result, may not take the actions necessary to restore communication. Such unrecognized communication interruptions may lead to prolonged periods during which data is not transmitted to the remote monitoring platform.

In various embodiments, the present disclosure enables reliable reconnection and communication continuity within wearable medical systems through an intelligent coordination of the wearable medical device, the mobile device executing a patient application, and one or more remote servers. The wearable medical device is configured to monitor a patient's physiological condition and provide therapy as needed, while the mobile device serves as a communication bridge to transmit patient data to the remote servers. In some examples, the wearable medical device can be configured for collecting patient data related to a cardiac condition. In some examples, the wearable medical device can be configured for delivering therapy upon detection of the cardiac condition. In some examples, the wearable medical device can be configured for at least one of collecting patient data related to a cardiac condition or delivering therapy upon detection of the cardiac condition. In some examples, the wearable medical device can be configured only for collecting patient data and does not deliver therapy. The patient application maintains the wireless connection with the wearable medical device and manages data synchronization and alert presentation. When the above-described system detects a lapse in communication, identified by an absence of data transmission for a defined period of inactivity, the remote servers automatically generate a communication directed to the patient's mobile device. This communication may instruct the patient to perform corrective actions such as restarting the patient application or reestablishing the wireless connection. In certain embodiments, the remote servers may analyze patient data to determine appropriate conditions for notification delivery, such as suppressing alerts during detected sleep periods to minimize disruption. The system thereby ensures that essential physiological data continues to flow reliably from the wearable medical device to the remote servers, enhancing continuity of monitoring and therapy readiness. Through automated detection, contextual communication, and patient-guided reconnection procedures, the disclosed system improves robustness, safety, and usability in remote cardiac monitoring and other wearable medical applications.

The present disclosure will now be described in detail with reference to the Figures.

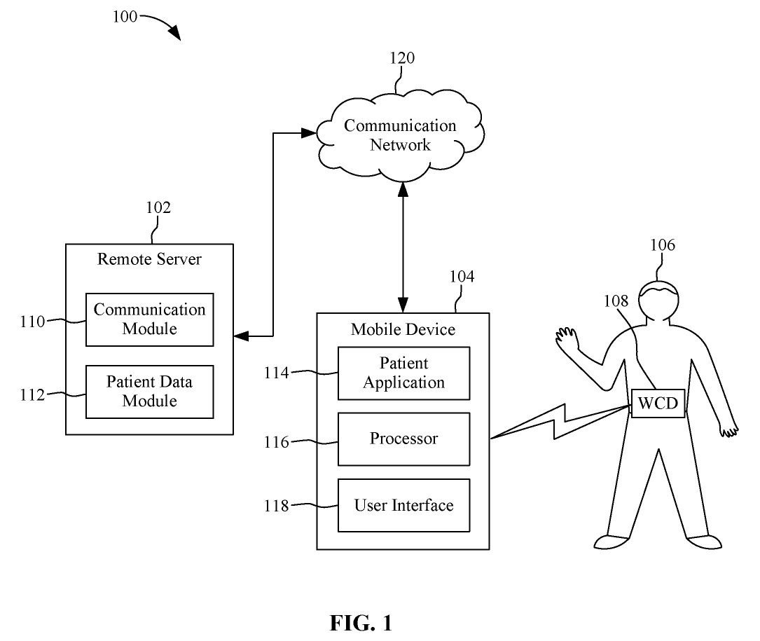

With reference now to FIG. 1, a medical system 100 for reestablishing communication between a wearable medical device and a mobile device is illustrated, in accordance with an embodiment of the present disclosure. The medical system 100 includes a remote server 102, a mobile device 104, a patient 106, a wearable cardioverter defibrillator (WCD) 108, which are communicatively coupled via a communication network 120. Within the context of the present disclosure, the term "medical system" may refer to an integrated hardware and software environment configured to facilitate continuous physiological monitoring, therapy delivery, and secure data exchange among distributed components. The medical system 100 establishes a cooperative operational framework in which the mobile device 104 communicates wirelessly with both the WCD 108 and the remote server 102 to ensure uninterrupted data transfer and therapy readiness for the patient 106. For purposes of clarity and brevity, a single remote server 102 is illustrated in FIG. 1; however, in other embodiments, multiple remote servers may be used to transmit, receive, store, or process medical data within the scope of the present disclosure. Accordingly, depending on the context, the remote server 102 may be referred to as one or more remote servers 102 throughout the disclosure.

In some embodiments, the medical system 100 is adapted for use in remote cardiac monitoring scenarios where the WCD 108 detects cardiac conditions associated with the patient 106 and delivers therapy to the patient 106 as required, while transmitting physiological and device status information, hereinafter referred to as "patient data", to the mobile device 104. The mobile device 104 operates as an intermediate communication node that establishes a wireless link with the WCD 108 and a data connection with the remote server 102 via the communication network 120. The medical system 100 ensures that patient data is periodically transmitted to the remote server 102 for remote monitoring, data storage, and alert management.

In some embodiments, the remote server 102 serves as the central data processing and communication management infrastructure within the medical system 100. The remote server 102 may include one or more cloud-hosted computing instances, on-premises servers within a healthcare network, or a combination thereof. In an exemplary embodiment, the remote server 102 may include the Kestra CareStation® remote data platform and/or a Salesforce® portal.

The remote server 102 includes functional components such as a communication module 110 and a patient data module 112. The communication module 110 is configured to generate and transmit communications to the mobile device 104 when an interruption in patient data transmission is detected. The patient data module 112 is configured to receive and process patient data transmitted from the mobile device 104, analyze transmission activity, and determine contextual states of the patient 106 such as rest, sleep, or activity levels based on physiological signals received from the WCD 108. The remote server 102 communicates with the mobile device 104 via the communication network 120 to maintain synchronization and continuity of patient monitoring.

The mobile device 104 includes a patient application 114, one or more processors 116 (referred to as a "processor 116" for brevity), and user interface 118, which collectively manage local communication, data relay, and patient interaction. In certain embodiments, the patient application 114 is remotely managed by the remote server 102, wherein the remote server 102 is configured to control, configure, and update operational aspects of the patient application 114. Such management may include, for example, provisioning application updates, modifying configuration parameters, enabling or disabling features, adjusting communication behaviors, and enforcing security or compliance policies, all without requiring direct user intervention.

In certain embodiments, the patient application 114 is associated with the WCD 108 such that the application is configured to communicate with the WCD 108 to receive physiological data, device usage information, or status information, and to relay such information to the remote server 102. In an embodiment, the patient application 114 may be installed on the mobile device 104 provided with the WCD 108 and may automatically transmit patient data to the remote server 102 at a plurality of transmission times or defined intervals. In another embodiment, the patient application 114 may transmit patient data to the remote server 102 in response to user or device events. Therefore, the patient application 114 operates as an integrated component of the medical system 100 to support clinician access to patient data and to engage the patient in their own cardiac recovery process.

The mobile device 104 may include, but is not limited to, a smartphone, a smartwatch, tablet, or any computing device capable of wireless communication and execution of a patient- specific application. The patient application 114 is configured to establish and maintain a wireless connection with the WCD 108, and to transmit patient data to the remote server 102 through the communication network 120. The processor 116 executes software instructions of the patient application 114 and manages data transfer, event logging, and reconnection protocols in response to communication interruptions. The user interface 118 includes a graphical or textual display, touchscreen elements, and audio outputs to provide notifications and instructions to the patient 106 when reconnection is required. In certain embodiments, the mobile device 104 is paired with the WCD 108 and uses a cellular, Wi-Fi, or other wireless communication channel to communicate with the remote server 102.

In some embodiments, the WCD 108 is a medical device configured to continuously monitor cardiac rhythms of the patient 106 and provide therapeutic intervention, such as electrical shock therapy, upon detection of life-threatening cardiac arrhythmias. The WCD 108 may include one or more sensors or electrodes configured to acquire physiological signals. The WCD 108 periodically generates patient data including physiological measurements, device status information, and operational metrics. The WCD 108 transmits the patient data to the mobile device 104 via a wireless communication link, such as Bluetooth, Bluetooth Low Energy (BLE), Wi-Fi Direct, or another short-range wireless protocol. The wireless communication link enables real- time data synchronization and continuous remote observation without the need for wired interfaces.

In some embodiments, the WCD 108 includes at least one therapy electrode for delivering an electric shock upon detection of a cardiac condition of the patient 106 based on patient data that includes an electrocardiogram (ECG) data of the patient 106. The at least one therapy electrode is carefully positioned on the patient's body to ensure maximum conductivity and effectiveness. The at least one therapy electrode may be made from conductive material that allows efficient transmission of electrical energy while minimizing skin irritation for long-term wear. The at least one therapy electrode may be connected to an energy source to generate and store the necessary electrical charge required for defibrillation. The energy source is typically a battery-powered capacitor system capable of delivering high-voltage shocks when needed. When the WCD 108 identifies a life-threatening arrhythmia, such as ventricular fibrillation (VF) or ventricular tachycardia (VT), the WCD 108 rapidly initiates the energy source and delivers the electric shock to the patient 106 via the at least one therapy electrode. The at least one therapy electrode releases carefully measured electrical pulse designed to reset the electrical activity of the heart of the patient 106 to restore a normal cardiac rhythm.

In an exemplary embodiment, the WCD 108 may be an ASSURE® WCD provided by Kestra Medical Technologies Inc., Kirkland WA (Kestra). However, other embodiments are not limited to using Kestra as the WCD provider without limitation. In embodiments, the WCD 108 and mobile device 104 are similar to those described in U.S. Patent No. 8,838,235, U.S. Patent No. 11,235,143, U.S. Patent No. 11,794,005, and U.S. Patent No. 11,950,174, all of which are incorporated by reference herein for all purposes.

The communication network 120 provides a secure data exchange channel between the mobile device 104 and the remote server 102. The communication network 120 may include any combination of public or private communication networks, including cellular networks (e.g., 3G, 4G, or 5G), wireless local area networks (e.g., Wi-Fi), or other data communication infrastructures, and may employ encrypted communication protocols such as HTTPS, TLS, or equivalents thereof to support secure data transmission. Through the communication network 120, the patient application 114 transmits patient data to the remote server 102 when a wireless communication link between the WCD 108 and the mobile device 104 is established. The remote server 102, in turn, may transmit communications, notifications, or instructions to the mobile device 104 when the remote server 102 determines that patient data has not been received for a defined period of inactivity.

In an exemplary operation, when the patient application 114 has been "silent" or inactive for a predetermined time period, the patient data module 112 is configured to cause a notification (e.g., a text message) to be transmitted to the patient's mobile device 104 via the communication module 110 of the remote server 102. In certain embodiments, the predetermined time period is longer than a duration required for the patient application 114 to enter a suspended state (e.g., 30 minutes). For example, an associated timer may be reset each time patient data is received from the mobile device 104. Under normal operating conditions (e.g., when the mobile device 104 is powered on and has cellular or Wi-Fi connectivity), the mobile device 104 may receive the notification even while the patient application 114 remains in the suspended state. The mobile device 104 may then notify the patient 106 of the notification and present the notification via the user interface 118.

In embodiments, a period of inactivity is defined to be greater than an interval between any two transmission times of a plurality of transmission times associated with normal operation of the patient application 114. During typical operation, the patient application 114 transmits the patient data to the remote server 102 at regular or recurring intervals, such as periodic check-in messages or event-driven uploads. By configuring the period of inactivity to exceed the expected interval between successive transmissions, the system 100 reliably distinguishes between routine transmission delays and abnormal communication interruptions, such as those caused by the patient application 114 entering a suspended state due to mobile operating system constraints

In exemplary and non-limiting implementations, the period of inactivity may be set to a fixed threshold, such as within a range of approximately 15 to 60 minutes, or may be dynamically computed based on one or more expected transmission intervals, historical transmission patterns, or application configuration parameters. For example, where the patient data is transmitted approximately every 15 minutes, the period of inactivity may be set to exceed 30 minutes, without being so limited. This approach reduces false detection of inactivity, avoids unnecessary patient notifications, and enables timely identification of true communication loss, thereby mitigating extended communication gaps between the WCD 108 and the remote server 102.

In another exemplary operation, the processor 116 of the mobile device 104 continuously monitors the status of the wireless connection between the patient application 114 and the WCD 108. When the wireless connection is active, the patient application 114 transmits patient data through the communication network 120 to the remote server 102, where it is processed by the patient data module 112. In an embodiment, when the patient data is not transmitted for a predefined duration (e.g., 5 minutes), the patient data module 112 of the remote server 102 automatically detects inactivity and generates a communication, via the communication module 110, configured to instruct the patient 106 to reestablish the wireless connection. In another embodiment, when the patient application 114 has been closed for a predetermined time (e.g., 5 minutes), the remote server 102 detects the event as "inactivity" and triggers a communication (e.g., an SMS text) to the mobile device 104. The communication is transmitted from the remote server 102 to the mobile device 104 via the communication network 120. Upon receipt of the communication, the patient application 114 presents the communication through the user interface 118, prompting the patient 106 to take corrective action such as restarting or relaunching the patient application 114 or repositioning the WCD 108 to restore connectivity.

In some embodiments, the processor 116 of the mobile device 104 is configured to execute program instructions stored within a memory (not shown) of the mobile device 104 to perform one or more operations associated with maintaining, monitoring, and reestablishing communication between the mobile device 104 and the WCD 108. The processor 116 is configured to determine whether patient data is currently being received from the WCD 108. In response to detecting that patient data is not being received for a threshold (or predefined) period, the processor 116 is further configured to initiate a diagnostic routine to confirm whether the wireless connection between the mobile device 104 and the WCD 108 is inactive or interrupted. Upon confirmation of the interruption, the processor 116 causes transmission of an inactivity signal through the communication network 120 to the remote server 102.

The remote server 102, in response to receiving the inactivity signal or determining that no patient data has been received for a predefined period, causes the communication module 110 to generate and transmit a communication to the mobile device 104. The communication includes one or more of, an instruction prompting the patient 106 to reestablish the wireless connection between the WCD 108 and the mobile device 104, a diagnostic notification indicating that the mobile device 104 or the WCD 108 may require user attention, or a contextual instruction specifying an action to restore data transmission. The communication may be generated as a message payload compatible with the patient application 114 and is transmitted through the communication network 120 using a secure messaging protocol. In an example, the communication may include an SMS text message.

Upon receipt of the communication, the processor 116 of the mobile device 104 is configured to process the received communication and cause display of a notification on the user interface 118. The notification provides a prompt, alert, or graphical indicator instructing the patient 106 to perform one or more actions to reestablish communication. In certain embodiments, the notification may include one or more of a text-based instruction, an icon-based indicator, an audible tone, and a vibration pattern, each configured to draw the patient's attention to a loss of communication.

In one example, the notification may include a text message containing a reminder to launch or relaunch the patient application 114. In another example, the text message may include an embedded link or selectable element that, when activated, causes the mobile device 104 to launch the patient application 114. In response to the text message, the patient 106 is expected to relaunch the patient application 114. In some embodiments, relaunching the patient application 114 may include an action by the patient 106 to close the patient application 114 and subsequently open it. Once opened, the patient application 114 may cause reestablishment of the communication link between the WCD 108 and the mobile device 104, thereby enabling patient data uploading from the WCD 108 to the remote server 102 via the mobile device 104.

In certain embodiments, transmission of the text-based notification resets a timer associated with monitoring application inactivity, such that an additional notification is transmitted upon expiration of the timer if the patient 106 does not launch or relaunch the patient application 114. In alternative embodiments, if the patient data module 112 executing on the remote server 102 does not receive patient data from the mobile device 104 within a predetermined time interval (e.g., within two minutes) following transmission of the notification, the patient data module 112 may transmit a subsequent notification. In some implementations, the subsequent notification may include additional guidance or instructions for launching or relaunching the patient application 114 on the mobile device 104. In certain embodiments, the medical system 100 is configured such that the WCD 108 periodically transmits status updates or data messages to the remote server 102 according to a defined schedule (e.g., after every 15 minute intervals). Each such update or "check- in" message received by the remote server 102 may cause the associated inactivity timer to be reset.

Further, the processor 116 is configured to detect a patient interaction with the user interface 118 in response to the notification, such as a user selection of a "Reconnect" control or initiation of a reconnection feature on the patient application 114. In response to the detected interaction, the processor 116 automatically executes a reconnection sequence to restore wireless communication with the WCD 108.

In embodiments, the patient data module 112 of the remote server 102 is configured to determine whether the patient 106 is in an inactive state, such as resting or sleeping, prior to the communication module 110 generating the communication. The patient data module 112 determines the inactive state based on previously received physiological signals from the WCD 108, which may include movement data, heart rate patterns, or posture information. In response to determining that the patient 106 is in an inactive state, the patient data module 112 is configured to cause suppression, prevention, or delay of the communication, thereby preventing unnecessary disturbance to the patient 106 during rest periods. The patient data module 112 further stores an inactivity record in association with a patient identifier, enabling retrospective analysis of patient activity and device communication performance.

In embodiments, the processor 116 is further configured to manage notification settings that govern how and when communications are presented via the user interface 118. The processor 116 may store a user profile in a memory of the mobile device 104, wherein the user profile defines parameters such as notification priority levels, notification delivery modes (e.g., visual, auditory, or haptic), and allowable time periods for notification presentation. The patient application 114, operating under control of the processor 116, accesses the user profile and applies the defined parameters to customize the presentation of notifications received from the remote server 102. In this manner, the processor 116 facilitates delivery of reconnection prompts in a context-aware manner that is consistent with patient comfort, usability considerations, and clinical safety requirements.

In an exemplary operation, the WCD 108 continuously monitors one or more physiological parameters of the patient 106 and transmits corresponding patient data to the mobile device 104. The mobile device 104, through execution of the patient application 114 by the processor 116, relays the patient data to the remote server 102 via the communication network 120. The patient data module 112 executing on the remote server 102 monitors incoming data streams and records associated transmission timestamps. When the communication module 110 determines that patient data has not been received for a threshold duration, the communication module 110 generates a notification or communication and transmits it to the mobile device 104. The mobile device 104, operating under control of the processor 116, presents the communication to the patient 106 via the user interface 118, thereby prompting and enabling the patient 106 to take action to reestablish communication with the WCD 108.

In embodiments, the processor 116 is further configured to verify successful reconnection following a user-initiated action. Upon determining that the WCD 108 has reestablished a wireless communication link with the mobile device 104, the processor 116 automatically transmits a reconnection acknowledgment to the remote server 102. The patient data module 112, upon receipt of the reconnection acknowledgment, updates a communication status associated with the patient 106 and resumes continuous data reception and processing. The closed- loop reconnection process described herein enhances system resiliency and facilitates timely restoration of data continuity, thereby mitigating the risk of monitoring gaps in patient cardiac data.

In embodiments, the communication module 110 is further configured to maintain a retry logic that defines conditions under which additional communications are transmitted when reconnection has not been achieved. The communication module 110 may transmit another (or second) communication to the mobile device 104 after a predetermined interval when the reconnection confirmation has not been received. In exemplary and non-limiting implementations, the predetermined interval may be within a range of approximately 5 to 30 minutes, may increase progressively between successive retry attempts, or may be dynamically determined based on one or more factors including prior transmission behavior, time of day, or patient activity patterns.

The processor 116 of the mobile device 104, upon receipt of the repeated communication, escalates a level of the notification presented via the user interface 118, for example, by increasing audio volume, activating a distinct visual alert, or triggering an automated voice instruction. The retry logic may further specify a maximum number of retry attempts, after which a service alert is generated for clinical personnel monitoring the patient 106 remotely.

The communication network 120 facilitates secure and reliable data transmission throughout the above-described operations. The processor 116 is configured to manage encrypted communication sessions with the remote server 102 using authentication protocols such as token- based authorization or digital certificates. Each message transmitted through the communication network 120 may include metadata such as a device identifier, timestamp, message type, and integrity checksum. The remote server 102 validates message authenticity and integrity upon receipt, ensuring regulatory compliance with data security standards applicable to medical data transmission.

In embodiments, the medical system 100 enables adaptive communication management. The processor 116 dynamically adjusts polling intervals, inactivity thresholds, and reconnection parameters based on network performance metrics or patient-specific behavioral patterns. For instance, when the patient 106 exhibits high mobility, the processor 116 may reduce inactivity thresholds to detect disconnections promptly. Conversely, during known resting periods, the processor 116 increases tolerance for communication delays to avoid unnecessary alerts. This adaptive control optimizes communication reliability while minimizing patient burden.

In an exemplary embodiment, the patient 106 wearing the WCD 108 may experience a temporary loss of wireless connectivity with the mobile device 104 while away from the communication range. The remote server 102, upon detecting the absence of incoming patient data for a threshold duration, causes the communication module 110 to generate and transmit a communication to the mobile device 104. When the patient 106 returns within the communication range, the processor 116 of the mobile device 104 receives the communication, displays a reconnection instruction on the user interface 118, and automatically executes a reconnection routine with the WCD 108. Upon successful reconnection, the processor 116 transmits an acknowledgment to the remote server 102, thereby restoring the continuity of patient data transmission and updating the monitoring system state.

In another exemplary embodiment, the remote server 102 and the mobile device 104 operate collaboratively or independently to implement an intelligent suppression mechanism. When the patient data module 112 determines, based on stored physiological data, that the patient 106 is in a sleep phase, the communication module 110 delays transmission of non-critical communications. Upon detecting that the patient 106 has resumed activity, the delayed communication is automatically transmitted. The processor 116, upon receiving the communication, presents it through the user interface 118, ensuring that reconnection guidance is provided only at clinically appropriate times without disrupting patient rest.

In some embodiments, the patient data module 112 can be configured to determine a sleep schedule associated with the patient 106. For example, the patient data module 112 can be configured to prevent communications and/or delay communications during times within the patient's sleep schedule. The patient's sleep schedule may not directly correspond with the patient 106 actually being asleep. However, the sleep schedule can be configured to prevent transmission of the communication during the entirety of a determined sleep schedule, such that even if the patient 106 is not asleep, the transmission of a communication is prevented during the sleep schedule. In some examples, the sleep schedule is determined using one or more computational models, including artificial intelligence models, algorithms, and/or machine learning models implemented within or accessible by the patient data module 112. Such models may be trained using supervised learning, unsupervised learning, semi-supervised learning, or combinations thereof, and may be further adapted to patient-specific behavior using techniques such as retrieval- augmented processing or incremental model updating. The models may leverage available data including, but not limited to, physiological signals (e.g., heart rate, heart rate variability, respiratory metrics), motion or posture data (e.g., accelerometer or position data), activity indicators (e.g., step counts or mobile device usage patterns), cloud-based health monitoring data, environmental context data, or other sensed or derived parameters to characterize behavioral patterns and estimate a probability that the patient 106 is asleep or inactive. In some examples, the models may be trained using historical patient data to determine and/or dynamically update the patient's sleep schedule. In some examples, the models may be trained using aggregated data from a population of patients and may be periodically or dynamically updated as additional data is received.

In embodiments, the medical system 100 may further include data analytics logic within the remote server 102 configured to evaluate communication trends across multiple patients. The communication module 110 may record reconnection frequency, average downtime duration, and signal strength indicators. The patient data module 112 aggregates such metrics for system optimization and predictive maintenance of device connectivity. The processor 116 may also locally store recent connectivity logs for diagnostic review by service technicians or healthcare providers through the patient application 114 interface.

Through the coordinated operation of the remote server 102, the mobile device 104, the WCD 108, and the communication network 120, the medical system 100 ensures robust reestablishment of communication following any interruption. The configuration described herein provides continuous data integrity, supports proactive reconnection through automated notifications, and minimizes the risk of unmonitored intervals in patient cardiac monitoring. By defining explicit functional roles for each component, the present disclosure enables reliable and secure restoration of data transmission pathways critical for real-time patient care.

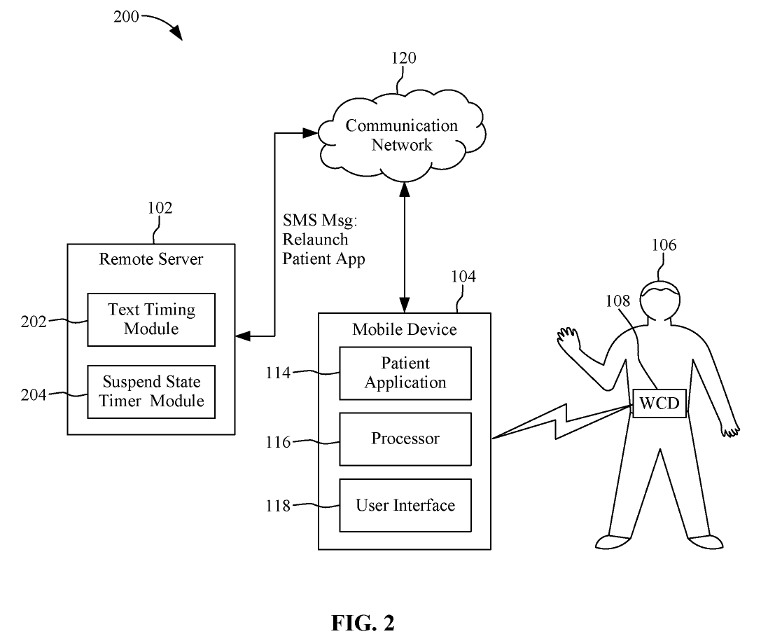

With reference now to FIG. 2, an exemplary environment 200 for implementing time- adaptive communication control in a medical system for reestablishing communication between a wearable medical device and a mobile device is illustrated, in accordance with an embodiment of the present disclosure. The environment 200 builds upon the medical system 100 described in FIG. 1 and introduces additional functionality within the remote server 102 to optimize delivery of communications based on patient activity and rest patterns.

In embodiments, the environment 200 includes the remote server 102, the mobile device 104, and the WCD 108, the patient 106, and the communication network 120. The above- mentioned components cooperate to ensure that communications intended to prompt reconnection between the WCD 108 and the mobile device 104 are delivered only during clinically appropriate and patient-tolerant time windows.

In embodiments, the remote server 102 includes at least a text timing module 202 and a suspend state timer module 204. The text timing module 202 and the suspend state timer module 204 may be implemented as components of the patient data module 112, as described with reference to FIG. 1, or may be implemented as modules separate from the patient data module 112 within the remote server 102.

The text timing module 202 is configured to determine permissible communication periods based on either configuration parameters defined during a fitting session by a healthcare provider, or analysis of physiological and behavioral data received from the WCD 108 through the patient data module 112. During the fitting session, the fitter or clinician may input patient-specific active hours (or non-sleeping hours) into the text timing module 202 via an administrative interface. In certain embodiments, the text timing module 202 is configured to analyze data received from the WCD 108 over a wear period to identify time intervals during a day, including day-of-week patterns, during which the patient 106 is likely to be active or awake. Based on this analysis, the text timing module 202 may selectively permit or suppress transmission of communications during identified time intervals in order to optimize notification effectiveness and reduce patient disruption. Alternatively, the text timing module 202 may autonomously learn patient activity cycles by analyzing heart-rate variability, posture information, or accelerometer signals transmitted by the WCD 108 over a defined observation period. Based on such data, the text timing module 202 generates a timing schedule that defines "awake periods" and "rest periods" for the patient 106.

The text timing module 202 communicates the generated timing schedule to the communication module 110 within the remote server 102. The communication module 110 uses the timing schedule as a gating condition for transmission of communications or text notifications to the mobile device 104. Although the present embodiment is described with reference to text- based notifications and a text timing module, the type of communication and the associated timing or control module may vary without departing from the scope of the present disclosure.

When the patient data module 112 or the processor 116 detects a communication interruption, the communication module 110 consults the timing schedule from the text timing module 202 to determine whether the current time falls within an allowable active period. When the time corresponds to a rest period, the communication module 110 temporarily suppresses, prevents, or delays the notification. When the next active period begins, the communication module 110 automatically releases the queued communication to the mobile device 104 through the communication network 120. In this manner, the environment 200 ensures that reconnection prompts are transmitted only when the patient 106 is awake or likely to respond, thereby minimizing unnecessary disturbance.

In an embodiment, the suspend state timer module 204 corresponds to a timer configured to track a duration during which the patient application 114 has not communicated with the remote server 102. If the patient application 114 remains inactive for a threshold period, the processor 116 of the mobile device 104, executing operating system or system-level software, may transition the patient application 114 into a suspended state, which may disrupt or disconnect the communication link between the mobile device 104 and the WCD 108.

In embodiments, the suspend state timer module 204 operates cooperatively with the text timing module 202 to control local notification timing and timer activation. The suspend state timer module 204 receives an input signal from the text timing module 202 indicating whether the patient 106 is in an active or rest state. In response to a "rest state" indication, the suspend state timer module 204 temporarily disables or pauses the timer function responsible for triggering outgoing communications related to reconnection reminders. When an "active state" indication is received, the suspend state timer module 204 reenables the timer, allowing standard notification and reconnection logic to proceed as described with respect to FIG. 1. This synchronization between the two modules prevents the medical system 100 from initiating non-critical notifications during periods when patient interaction is unlikely or undesired.

During exemplary operation of the environment 200, the WCD 108 continuously transmits patient data directly to the mobile device 104 and further to the remote server 102 (via the mobile device 104). The patient data module 112 analyzes the received data to identify daily activity patterns and forwards summarized metrics to the text timing module 202. The text timing module 202 updates its stored timing schedule, and accordingly refines the definition of patient- specific awake intervals over time. When the processor 116 of the mobile device 104 detects that patient data has not been received from the WCD 108 for a threshold duration, the processor 116 causes transmission of an inactivity signal to the remote server 102. The communication module 110, prior to sending a reconnection communication, queries the text timing module 202 for a timing authorization. If the current time is within an active period, the communication is immediately transmitted; otherwise, it is queued until the suspend state timer module 204 reenables its delivery window.

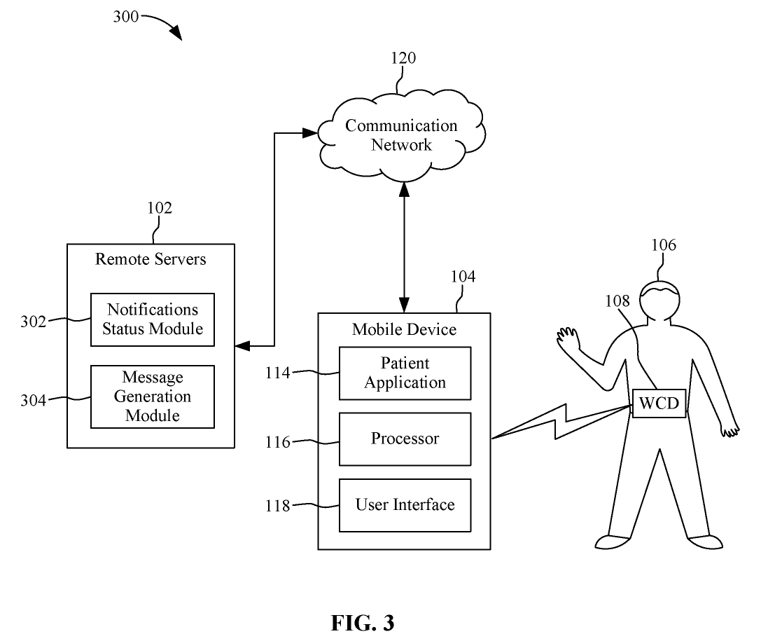

With reference now to FIG. 3, an environment 300 for detecting notification settings and transmitting communication prompts to a patient device is illustrated, in accordance with an embodiment of the present disclosure. The environment 300 includes the remote server 102, the mobile device 104, the patient 106, the WCD 108, and the communication network 120. The remote server 102 further includes a notification status module 302 and a message generation module 304. The environment 300 establishes a cooperative operational framework that enables detection of whether notifications for the patient application 114 on the mobile device 104 are turned off and, in response, initiates a text-based or multimedia message prompting the patient 106 to enable notifications. FIG. 3 is described in conjunction with previous figures.

Within the context of the present disclosure, the term environment 300 may refer to an integrated system of hardware and software components configured to perform communication verification, notification monitoring, and message prompting functions associated with a wearable medical monitoring system. The environment 300 operates in conjunction with the WCD 108 and the mobile device 104 to ensure continuous patient engagement and uninterrupted data transmission between the mobile device 104 and the remote server 102 through the communication network 120. The environment 300 is further configured to detect user-controlled mobile device settings that may hinder communication reliability, such as disabled notification permissions, and to automatically generate remedial guidance to restore operational conditions required for effective therapy monitoring.

In certain embodiments, the notification status module 302 and the message generation module 304 may be implemented as separate modules within the remote server 102 or may be integrated as components of the patient data module 112. In a particular implementation, the message generation module 304 may be included as part of the communication module 110. In another implementation, the message generation module 304 may operate in coordination with the communication module 110 to generate and transmit communications or messages. The remote server 102 is configured to receive periodic data uploads from the patient application 114, including physiological data obtained from the WCD 108 and metadata describing the operational state of the mobile device 104 and the patient application 114, such as connectivity parameters and notification status.

The notification status module 302 is configured to detect whether the notification setting of the patient application 114 on the mobile device 104 is enabled or disabled. The notification status module 302 may perform such detection through one or more methods. In an exemplary embodiment, the notification status module 302 receives, within each data upload transmitted from the patient application 114, a notification status indicator that specifies whether notification permissions on the mobile device 104 are currently active. The notification status module 302 analyzes the notification status indicator and determines whether the patient 106 has turned off notifications for the patient application 114 through the settings interface of the mobile device 104. In alternate embodiments, the notification status module 302 is configured to query the patient application 114 via the communication network 120 to obtain the current notification setting, thereby enabling active verification without requiring patient input. The notification status module 302 may further record the notification setting status in association with a patient identifier for ongoing compliance tracking and operational analytics.

In embodiments, the message generation module 304 is configured to generate and transmit communication prompts to the mobile device 104 in response to detecting that the notification setting of the patient application 114 is disabled. The message generation module 304 operates in coordination with the communication module 110 of the remote server 102 to compose and transmit a communication which may include a text message, a multimedia message, a push notification, an in-application prompt, or another electronically deliverable recommendation, or control that guides the patient 106 to enable notifications on the mobile device 104. The message generation module 304 may include message formatting and delivery logic to ensure compatibility with one or more communication mechanisms supported by the communication network 120 and the mobile device 104, including Short Message Service (SMS), Multimedia Messaging Service (MMS), push notification frameworks, application-level messaging channels, or equivalent protocols. In certain embodiments, the message generation module 304 generates a multimedia message containing graphical content, animations, or interactive elements, such as step-by-step illustrations, visual depictions of the mobile device settings interface, or selectable controls that, when activated, trigger navigation to a notification settings screen. In some implementations, the communication may further initiate or facilitate a remote-triggered action on the mobile device 104, such as launching the patient application 114 or opening a system settings interface, thereby simplifying patient interaction and increasing the likelihood of compliance.

In an exemplary embodiment, the mobile device 104 executes the patient application 114 via the processor 116. The patient application 114 includes program instructions configured to locally detect the notification permission state of the mobile device 104. For example, the patient application 114 may determine whether the mobile operating system has enabled or disabled alerts, banners, sounds, or other notification modalities associated with the patient application 114. The patient application 114 embeds a corresponding notification status flag within each data upload transmitted to the remote server 102 via the communication network 120.

In alternative embodiments, where direct access to notification permission information is unavailable, restricted, or unreliable due to operating system limitations or privacy constraints, the notification status may be inferred indirectly. For example, the patient application 114 and/or the remote server 102 may initiate one or more silent or low-visibility probe communications using different delivery mechanisms, such as application-level messages, push notifications, text messages, or other communication channels, and may monitor for acknowledgments, responses, or delivery confirmations. Based on which probe communications are successfully received or acknowledged by the mobile device 104, the system may infer an effective notification permission state and select a preferred communication delivery method. The inferred notification status may likewise be encoded in a notification status flag and transmitted to the remote server 102 for use in subsequent message generation and delivery decisions

In response to receiving the data upload, the notification status module 302 verifies the embedded flag and determines whether the patient application 114 is permitted to present notifications on the user interface 118. .

The user interface 118 of the mobile device 104 is configured to present information, alerts, and interactive options associated with notification status management. In embodiments, when the patient application 114 receives an SMS message, an MMS message, a push notification, or another electronically deliverable message generated by the message generation module 304, the user interface 118 displays the message content, including text instructions or graphical representations guiding the patient 106 through the steps necessary to enable notifications.

In some implementations, the user interface 118 may further provide one or more direct access controls that launch the settings interface of the mobile device 104 to allow immediate adjustment of notification permissions. In addition, in certain embodiments, receipt of a communication or remote command may trigger one or more operating-system-permitted actions on the mobile device 104, such as launching the patient application 114, bringing the patient application 114 to a foreground state, initiating an inter-application interaction, or opening a system configuration interface associated with notification or application behavior. Such actions may be performed in accordance with operating system security policies and user permissions and may simplify patient interaction while increasing the likelihood of restoring proper notification and communication functionality.

The processor 116 detects patient input through the user interface 118 and records an acknowledgment signal indicating that the patient 106 has viewed or acted upon the message. The acknowledgment signal may then be transmitted to the remote server 102 through the communication network 120 for logging and verification by the notification status module 302. In certain embodiments, when an acknowledgment signal is not received within a predetermined time interval, the remote server 102 is configured to initiate one or more alternate notification actions. For example, a subsequent communication may be transmitted using a different delivery mechanism than a prior communication, such as transitioning from a text-based message (e.g., SMS) to a push notification, from a push notification to an automated voice message or audible alert, or from an application-level message to a system-level notification, without limitation. In addition, when device-based notification attempts do not result in reconnection or acknowledgment, the remote server 102 may initiate communications using alternative communication modalities, including electronic mail messages, automated or manual telephone calls, voice messages, or physical correspondence, such as postal or courier-delivered letters. In some implementations, the alternate notification actions may further include escalation of a notification priority or generation of an alert for clinical personnel. Such alternate notification actions increase the likelihood that the patient 106 receives and responds to the communication when an initial notification attempt is unsuccessful.

In an exemplary operational flow, the WCD 108 continuously monitors physiological data of the patient 106 and transmits the data to the mobile device 104. The patient application 114, through execution by the processor 116, uploads the physiological data along with metadata including the notification status indicator to the remote server 102 via the communication network 120. The notification status module 302 analyzes the uploaded data and identifies that the notification status indicator is set to "off." In response, the message generation module 304 generates a text message containing the instruction "Please enable notifications for your Patient App to ensure continuous monitoring" and a hyperlink to the mobile device settings. The communication module 110 transmits the generated message to the mobile device 104. The user interface 118 presents the received message, and the patient 106 selects the hyperlink, causing the mobile device 104 to open the settings interface for enabling notifications. The notification status module 302 subsequently verifies, in a later upload, that the notification setting has been restored to the "on" state.

In other embodiments, the message generation module 304 generates an MMS message containing graphical content to prompt the patient 106 to relaunch the patient application 114 or modify the notification configuration. The graphical content may include one or more images or animated sequences depicting the patient application 114 and the steps required to access the notification permissions of the mobile device 104. The message generation module 304 may further include logic to embed hyperlinks or deep links within the MMS message that, when activated by the patient 106, launch the patient application 114 directly or open the specific settings menu for notification control. The remote server 102 records the time and status of each sent message and may suppress repeated messages once the notification status module 302 verifies that notifications have been successfully reenabled.

Through the coordinated operation of the notification status module 302, the message generation module 304, the patient application 114, and the processor 116, the environment 300 ensures that communication pathways between the WCD 108, the mobile device 104, and the remote server 102 remain functionally active and that the patient 106 remains promptly informed of system requirements. The described configuration provides an automated mechanism to detect notification deactivation, generate patient-specific instructional content, and restore interactive communication channels essential for the ongoing monitoring and therapeutic reliability of the wearable medical system.

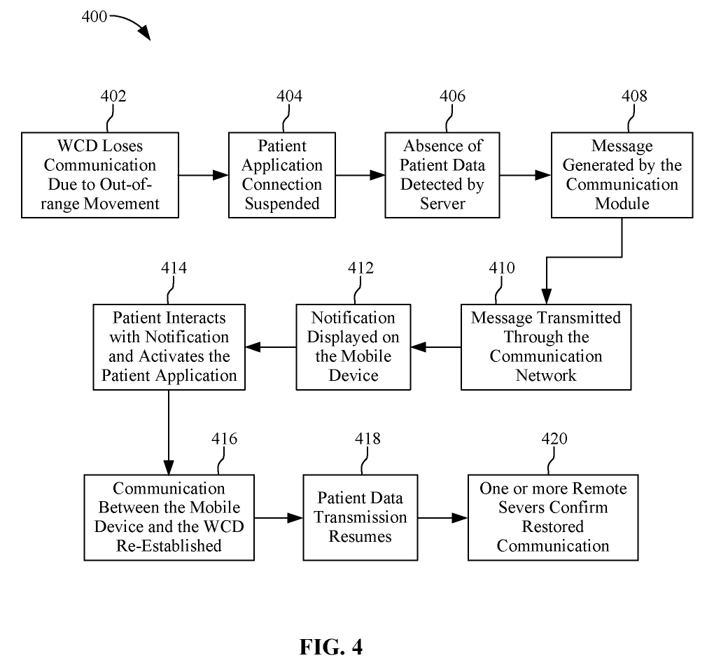

With reference now to FIG. 4, a flowchart 400 illustrates an exemplary operational sequence within the environment 200 showing reestablishment of communication between the WCD 108 and the mobile device 104, in accordance with an embodiment of the present disclosure. The flowchart 400 represents a high-level depiction of the reconnection logic executed cooperatively by the communication module 110, the patient application 114, and the processor 116 during a temporary interruption of patient data transmission. The flowchart 400 further demonstrates the closed-loop interaction among the distributed system components that ensures uninterrupted monitoring and restoration of patient data continuity for the patient 106.

At operation 402, the WCD 108 loses communication with the mobile device 104 as a result of the patient 106 moving beyond the effective communication range of the short-range wireless interface. The WCD 108 continues to monitor physiological parameters locally; however, transmission of patient data to the mobile device 104 through the communication network 120 becomes temporarily interrupted. This interruption represents a condition in which the monitoring continuity of the medical system 100 is momentarily compromised, thereby initiating a series of reconnection management steps.

At operation 404, the patient application 114 executing on the mobile device 104, through operation of the processor 116, detects that patient data is not currently being received from the WCD 108. The processor 116 identifies a suspended state of communication, updates a local status record within the memory of the mobile device 104, and awaits restoration of wireless connectivity. The patient application 114 may log the timestamp of the detected suspension and generate a corresponding status flag indicating the inactive communication state.

At operation 406, one or more remote servers 102 detect the absence of patient data uploaded from the mobile device 104 for a predefined duration. The patient data module 112 analyzes incoming transmission timestamps and determines that a data gap has occurred. In response to this detection, the communication module 110 is triggered to perform a communication restoration routine as part of the automated monitoring protocol. The one or more remote servers 102 thereby act as supervisory infrastructure ensuring that loss of data transmission is identified in real time.

At operation 408, the communication module 110 of the one or more remote servers 102 generates a message configured to prompt the patient 106 to take corrective action to reestablish communication. The message may be formatted as a text-based instruction, graphical indicator, or notification payload compatible with the patient application 114. The communication module 110 includes logic for message generation, encoding, and secure delivery through the communication network 120. In embodiments, the message generation process further references contextual data from the patient data module 112, ensuring that the message is transmitted only during suitable time intervals consistent with patient comfort and clinical safety.

At operation 410, the message generated by the communication module 110 is transmitted to the mobile device 104 through the communication network 120. The communication network 120 may employ cellular or Wi-Fi data channels configured with secure transmission protocols to preserve confidentiality and integrity of the message. The one or more remote servers 102 maintain a record of each message transmission, including metadata such as a timestamp, message type, and delivery status confirmation, thereby enabling traceability and compliance with medical communication standards.

At operation 412, the processor 116 of the mobile device 104 receives the message from the one or more remote servers 102 and processes the message within the patient application 114. The patient application 114, under execution control of the processor 116, causes a corresponding notification to be displayed on the user interface 118. The notification presents a text or graphical alert indicating that data transmission from the WCD 108 has been interrupted and provides an instruction to the patient 106 to relaunch or reopen the patient application 114 to reestablish connectivity.

At operation 414, the patient 106 interacts with the notification displayed through the user interface 118 and activates the patient application 114 in response to the instruction. The processor 116, upon detecting this user interaction, initiates a reconnection procedure within the patient application 114. The reconnection procedure may include restarting the local wireless interface, performing a device discovery process, and re-pairing with the WCD 108. The patient application 114 thereby resumes its role as the intermediate communication node facilitating secure data relay to the one or more remote servers 102.

At operation 416, the processor 116 successfully reestablishes communication between the mobile device 104 and the WCD 108. Once connectivity is restored, the patient application 114 confirms readiness to receive and transmit patient data, and the local suspended status record is updated to indicate an active communication state. The WCD 108, upon detection of an available communication path, resumes regular data exchange with the mobile device 104 without requiring manual device intervention.

At operation 418, transmission of patient data resumes. The WCD 108 transmits physiological signals and device status information to the mobile device 104, which in turn relays the data through the communication network 120 to the one or more remote servers 102. The patient data module 112 of the one or more remote servers 102 resumes continuous monitoring and storage of the received data. This restoration of streaming activity ensures that the medical system 100 maintains data integrity and continuity of patient monitoring without significant loss of information.

At operation 420, the one or more remote servers 102 confirm the successful restoration of communication. The patient data module 112 detects renewed receipt of patient data and clears the inactivity condition previously recorded. The communication module 110 may optionally transmit an acknowledgment or verification message to the mobile device 104 indicating that normal operation has been restored. This final operation closes the communication loop, confirming that the cooperative actions of the mobile device 104, the WCD 108, and the one or more remote servers 102 have effectively reestablished continuous patient data transmission.

In exemplary embodiments, the operations illustrated in the flowchart 400 may be automatically repeated whenever the processor 116 of the mobile device 104 or the communication module 110 of the one or more remote servers 102 detect a communication interruption exceeding a threshold period. The communication network 120 provides the secure bidirectional link through which status, messages, and data are exchanged. Through this closed-loop reconnection process, the medical system 100 ensures resilience, maintains therapy readiness of the WCD 108, and safeguards continuous cardiac monitoring of the patient 106.

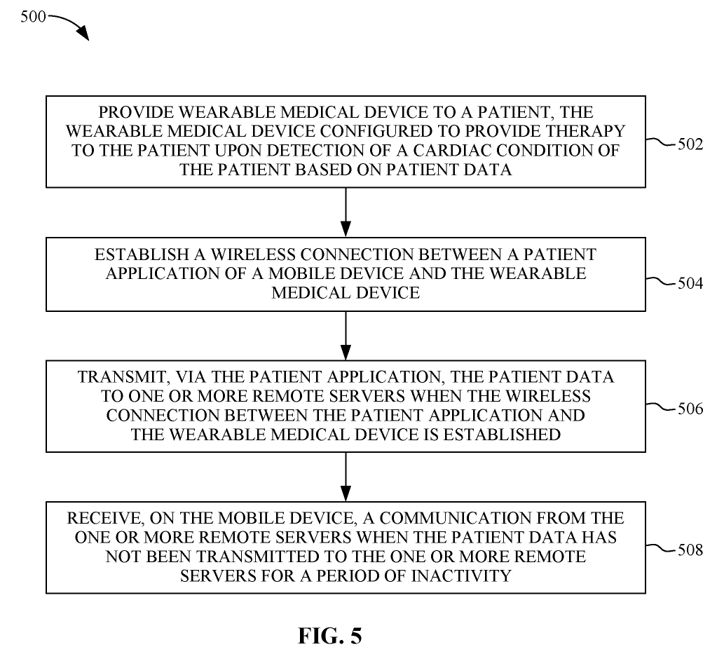

Referring now to FIG. 5, illustrated is a method 500 for reestablishing communication in a wearable medical system, in accordance with an embodiment of the present disclosure. The method 500 may be implemented by the medical system 100, including coordinated execution by the processor 116 of the mobile device 104, the communication module 110 and the patient data module 112 of the one or more remote servers 102, and the control logic embedded within the WCD 108. The method 500 may be executed as a sequence of operations defined by computer- executable program instructions stored in one or more memories of the mobile device 104 or the one or more remote servers 102, and is explained in conjunction with the system elements previously described with reference to FIGS. 1-4.

At step 502, the method 500 includes providing the WCD 108 to the patient 106. The WCD 108 is configured to detect cardiac conditions of the patient 106 and to deliver therapeutic intervention in response to detection of a life-threatening arrhythmia. The WCD 108 includes one or more physiological sensors configured to continuously acquire cardiac signals, motion data, and posture information, which collectively define patient data. In embodiments, the WCD 108 is fitted to the patient 106 by a clinician or fitter, and is linked to a patient profile stored within the one or more remote servers 102 to enable ongoing monitoring and therapy readiness verification.

At step 504, the method 500 includes establishing a wireless connection between the patient application 114 executing on the mobile device 104 and the WCD 108. The processor 116 of the mobile device 104 initiates a pairing and handshake routine using a short-range wireless protocol such as BLE or Wi-Fi Direct. Upon successful authentication, the patient application 114 and the WCD 108 exchange connection parameters, encryption keys, and synchronization timestamps to enable secure bidirectional communication. The wireless connection thereby forms the primary data pathway for transmission of physiological information and device status from the WCD 108 to the mobile device 104. In embodiments, the patient application 114 maintains periodic link-status polling and automatically retries reconnection upon transient signal degradation, ensuring persistent connectivity during patient mobility.

At step 506, the method 500 includes transmitting, via the patient application 114, the patient data to the one or more remote servers 102 when the wireless connection between the patient application 114 and the WCD 108 is established. The processor 116, under control of the patient application 114, formats the received patient data into secure message payloads and transmits the payloads through the communication network 120 to the one or more remote servers 102. The communication network 120 may include one or more cellular or Wi-Fi infrastructures configured for encrypted data transmission using protocols such as HTTPS or TLS. The patient data module 112 of the one or more remote servers 102 receives and processes the patient data, performs validation of message integrity, and stores the data within a secure patient record repository for remote monitoring and analysis. In embodiments, the patient data module 112 further derives contextual parameters such as activity state, rest periods, or device wear compliance, enhancing clinical visibility into patient status.

At step 508, the method 500 includes receiving, on the mobile device 104, a communication from the one or more remote servers 102 when the patient data has not been transmitted to the one or more remote servers 102 for a period of inactivity. The communication module 110 of the one or more remote servers 102 monitors data reception timestamps and determines that patient data has not been received for a predefined threshold duration. In response, the communication module 110 generates a communication addressed to the mobile device 104, where the communication includes an instruction to the patient 106 to reestablish the wireless connection between the WCD 108 and the patient application 114. The communication is transmitted through the communication network 120 and processed by the patient application 114 upon receipt. The processor 116 of the mobile device 104 causes display of a corresponding notification through the user interface 118, providing a reconnection prompt to the patient 106. Upon interaction by the patient 106 with the displayed notification, the processor 116 executes a reconnection sequence to restore communication with the WCD 108.

In embodiments, the method 500 may further include additional operations that enhance the reliability and contextual awareness of the reconnection process. For example, the patient data module 112 may determine, based on previously received physiological data, whether the patient 106 is in an inactive or sleep state and may accordingly suppress or delay transmission of the communication generated by the communication module 110. Similarly, the processor 116 may be configured to manage user-specific notification preferences stored within the mobile device 104, such that notifications are presented only during permissible time windows. These enhancements maintain patient comfort while ensuring timely restoration of data transmission when clinically appropriate.

In exemplary operation, the method 500 provides an integrated, automated framework through which the medical system 100 maintains continuous data communication among the WCD 108, the mobile device 104, and the one or more remote servers 102. The cooperative execution of the steps defined in the method 500 minimizes communication downtime, ensures immediate detection of transmission interruptions, and facilitates user-guided reconnection supported by intelligent server-side logic. By combining hardware, software, and network-based elements into a unified operational sequence, the method 500 enables consistent, secure, and resilient patient monitoring in wearable medical systems.

Accordingly, the embodiments described in the present disclosure improve reliability and continuity of communication in wearable medical systems by enabling a patient application to automatically or by user-assistance reestablish a wireless communication link with a wearable medical device after the patient application transitions from a suspended state to an active or other operational state. By facilitating timely restoration of connectivity, the disclosed systems and methods enable physiological, event, and usage data to be uploaded to a remote monitoring platform (or server) in a timely manner, thereby reducing extended communication gaps that may otherwise occur without patient awareness. The disclosed techniques further enhance system resiliency by addressing mobile operating system constraints, improving data continuity for clinical monitoring, and reducing reliance on manual intervention, thereby supporting robust and scalable remote patient monitoring.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

The descriptions of the various embodiments of the present disclosure have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Other embodiments include combinations and sub-combinations of features described or shown in the drawings herein, including for example, embodiments that are equivalent to: providing or applying a feature in a different order than in a described embodiment, extracting an individual feature from one embodiment, and inserting such feature into another embodiment; removing one or more features from an embodiment; or both removing one or more features from an embodiment and adding one or more features extracted from one or more other embodiments while providing the advantages of the features incorporated in such combinations and sub- combinations. As used in this paragraph, feature or features can refer to the structures and/or functions of an apparatus, article of manufacture or system, and/or the steps, acts, or modalities of a method.

These and other changes can be made to the embodiments in light of the above- detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

In construing the claims of this document, the inventor(s) invoke 35 U.S.C. § 112(f) only when the words "means for" or "steps for" are expressly used in the claims. Accordingly, if these words are not used in a claim, then that claim is not intended to be construed by the inventor(s) in accordance with 35 U.S.C. § 112(f).

What is claimed is:

1. A medical system comprising:
a wearable medical device configured for at least one of collecting patient data related to detection of a cardiac condition or providing therapy to a patient upon detection of the cardiac condition;
one or more remote servers; and
a mobile device comprising at least one processor and a user interface, the mobile device configured to be in communication with the wearable medical device and the one or more remote servers, the at least one processor configured to:
establish a wireless connection between the mobile device and the wearable medical device;
transmit, via the mobile device, the patient data to the one or more remote servers when the wireless connection between the mobile device and the wearable medical device is established; and
receive a communication from the one or more remote servers when the patient data has not been transmitted to the one or more remote servers for a period of inactivity,
wherein the communication is configured to provide one or more instructions to the patient to perform one or more actions to reestablish the wireless connection between the mobile device and the wearable medical device.

2. The medical system of claim 1, wherein a user interface of the mobile device is configured to display the communication, and wherein the one or more instructions include instructing the patient to perform the one or more actions to restart a patient application of the mobile device.

3. The medical system of claim 1, wherein the mobile device is configured to transmit the patient data at a plurality of transmission times.

4. The medical system of claim 3, wherein the period of inactivity is greater than an interval between any two transmission times of the plurality of transmission times.

5. The medical system of claim 1, wherein the one or more remote servers are configured to prevent transmission of the communication based on the patient data.

6. The medical system of claim 5, wherein the one or more remote servers include a patient data module configured to determine a sleep schedule based on the patient data, and wherein transmission of the communication is prevented when the patient is in a sleep state.

7. The medical system of claim 1, wherein the at least one processor is further configured to receive a second communication from the one or more remote servers when a notification setting of the mobile device is in an off state, wherein the patient data includes the notification setting of the mobile device, and wherein the second communication is configured to provide one or more additional instructions to the patient to perform one or more additional actions to change the notification setting to an on state.

8. The medical system of claim 1, wherein the wearable medical device comprises at least one therapy electrode in communication with an energy source, the at least one therapy electrode configured to deliver a shock to the patient based on the patient data.

9. A method for reestablishing a wireless connection between a mobile device and a wearable medical device, the method comprising:
providing the wearable medical device to a patient, the wearable medical device configured for at least one of collecting patient data related to detection of a cardiac condition or providing therapy to a patient upon detection of the cardiac condition;
establishing the wireless connection between the mobile device and the wearable medical device;
transmitting, via the mobile device, the patient data to one or more remote servers when the wireless connection between the mobile device and the wearable medical device is established; and
receiving, on the mobile device, a communication from the one or more remote servers when the patient data has not been transmitted to the one or more remote servers for a period of inactivity,
wherein the communication is configured to provide one or more instructions to the patient to perform one or more actions to reestablish the wireless connection between the mobile device and the wearable medical device.

10. The method of claim 9, wherein the communication is displayed on a user interface of the mobile device, and wherein the one or more instructions include instructing the patient to perform the one or more actions to restart a patient application of the mobile device.

11. The method of claim 9, wherein the mobile device is configured to transmit the patient data at a plurality of transmission times.

12. The method of claim 11, wherein the period of inactivity is greater than an interval between any two transmission times of the plurality of transmission times.

13. The method of claim 9, wherein the one or more remote servers are configured to prevent transmission of the communication based on the patient data.

14. The method of claim 13, wherein the one or more remote servers include a patient data module configured to determine a sleep schedule based on the patient data, and wherein transmission of the communication is prevented when the patient is in a sleep state.

15. The method of claim 9, the method further comprising receiving, on the mobile device, a second communication from the one or more remote servers when a notification setting of the mobile device is in an off state, wherein the patient data includes the notification setting of the mobile device, and wherein the second communication is configured to provide one or more additional instructions to the patient to perform one or more additional actions to change the notification setting to an on state.

16. A non-transitory computer readable medium, encoded with instructions stored thereon for reestablishing a wireless connection between a mobile device and a wearable medical device, that when executed by at least one computing device, cause the at least one computing device to perform operations for reestablishing the wireless connection, the operations comprising:
establishing the wireless connection between the mobile device and the wearable medical device, the wearable medical device configured for at least one of collecting patient data related to detection of a cardiac condition or providing therapy to a patient upon detection of the cardiac condition;
transmitting, via the mobile device, the patient data to one or more remote servers when the wireless connection between the mobile device and the wearable medical device is established; and
receiving, on the mobile device, a communication from the one or more remote servers when the patient data has not been transmitted to the one or more remote servers for a period of inactivity,
wherein the communication is configured to provide one or more instructions to the patient perform one or more actions to reestablish the wireless connection between the mobile device and the wearable medical device.

17. The non-transitory computer readable medium of claim 16, wherein the communication is displayed on a user interface of the mobile device, and wherein the one or more instructions include instructing the patient to perform the one or more actions to restart a patient application of the mobile device.

18. The non-transitory computer readable medium of claim 16, wherein the mobile device is configured to transmit the patient data at a plurality of transmission times, and wherein the period of inactivity is greater than an interval between any two transmission times of the plurality of transmission times.

19. The non-transitory computer readable medium of claim 16, wherein the one or more remote servers are configured to prevent transmission of the communication based on the patient data, wherein the one or more remote servers include a patient data module configured to determine a sleep schedule based on the patient data, and wherein the transmission of the communication is prevented when the patient is in a sleep state.

20. The non-transitory computer readable medium of claim 19, the operations further comprising receiving a second communication from the one or more remote servers when a notification setting of the mobile device is in an off state, wherein the patient data includes the notification setting of the mobile device, and wherein the second communication is configured to provide one or more additional instructions to the patient to perform one or more additional actions to change the notification setting to an on state.
